(19) United States
(12) Patent Application Publication
(10) Pub. No.: US 2022/0287575 A1
(43) Pub. Date: Sep. 15, 2022

(54) SPHYGMOMANOMETER

(71) Applicant: OMRON HEALTHCARE CO., LTD., Muko-shi (JP)

(72) Inventors: Katsunori KONDO, Kyoto (JP); Shingo YAMASHITA, Kyoto (JP)

(73) Assignee: OMRON HEALTHCARE CO., LTD., Muko-shi (JP)

(21) Appl. No.: 17/827,498

(22) Filed: May 27, 2022

Related U.S. Application Data
(63) Continuation of application No. PCT/JP2020/045323, filed on Dec. 4, 2020.

(30) Foreign Application Priority Data
Dec. 9, 2019 (JP) 2019-222303

(51) Int. Cl. A61B 5/0225; A61B 5/00; A61B 5/022

(52) U.S. Cl. CPC A61B 5/0225; A61B 5/6824; A61B 5/02233; A61B 5/742

(57) ABSTRACT

A sphygmomanometer of the present invention is a sphygmomanometer having an automatic blood pressure measurement mode for automatically starting blood pressure measurement according to a predetermined schedule. The sphygmomanometer includes a sphygmomanometer main body configured to be mounted on a measured part and a plurality of operation units to which instructions different from each other are input, the operation units being provided on the sphygmomanometer main body. The operation unit includes an automatic blood pressure measurement mode operation unit to which an automatic blood pressure measurement mode instruction is input for switching a mode to the automatic blood pressure measurement mode. The sphygmomanometer includes a restriction unit that restricts a function of the operation unit when the sphygmomanometer is set in the automatic blood pressure measurement mode as a result of the automatic blood pressure measurement mode operation unit being operated.

### CROSS-REFERENCE TO RELATED APPLICATIONS

This is a continuation application of International Application No. PCT/JP2020/045323, with an International filing date of Dec. 4, 2020, which claims priority of Japanese Patent Application No. 2019-222303 filed on Dec. 9, 2019, the entire content of which is hereby incorporated by reference.

### TECHNICAL FIELD

The present invention relates to a sphygmomanometer, and more particularly to a sphygmomanometer having an automatic (during-sleep) blood pressure measurement mode.

### BACKGROUND ART

In general, blood pressure measurement is preferably performed in the same time zone every day in order to reliably check blood pressure of a subject. Since the blood pressure measurement needs to be performed when the subject spends his/her daily life, it is preferable to perform the blood pressure measurement using a sphygmomanometer that does not give the subject a physical burden as much as possible. To satisfy this requirement, Patent Document 1 (WO 2012/018029 A) discloses a wrist-type sphygmomanometer that automatically performs blood pressure measurement at a preset time. With the above-described wrist-type sphygmomanometer, for example, even in nighttime when the subject is sleeping, the blood pressure measurement can be automatically performed without disturbing the sleep of the subject.

### SUMMARY OF THE INVENTION

In contrast, the subject during sleep easily changes a measurement posture, particularly a direction of a wrist, for example, by turning over. At this time, a switch provided on a wrist-type sphygmomanometer main body may be pressed against bedding or the like and erroneously operated, by which automatic blood pressure measurement may not be performed.

The present invention has been made to solve the above-described problem, and an object of the present invention is to provide a sphygmomanometer capable of avoiding a situation in which automatic blood pressure measurement is not correctly performed by unintended operation when an automatic blood pressure measurement mode is set.

To achieve the object, a sphygmomanometer of the present disclosure has an automatic blood pressure measurement mode for automatically starting blood pressure measurement according to a predetermined schedule, the sphygmomanometer comprising:

a sphygmomanometer main body configured to be mounted on a measured part; and

a plurality of operation units to which instructions different from each other are input, the plurality of operation units being provided on the sphygmomanometer main body, wherein

the operation unit includes an automatic blood pressure measurement mode operation unit to which an automatic blood pressure measurement mode instruction is input for switching a mode to the automatic blood pressure measurement mode, and

the sphygmomanometer includes a restriction unit that restricts a function of the operation unit when the sphygmomanometer is set in the automatic blood pressure measurement mode as a result of the automatic blood pressure measurement mode operation unit being operated.

In the present description, the “operation unit” typically refers to a switch provided on the main body of the sphygmomanometer, and receives switching on by the user (mainly a subject) as an instruction. Note that operation itself of the sphygmomanometer may be performed by an instruction from a smartphone or the like existing outside the sphygmomanometer via wireless communication.

To “restrict” a function of the operation unit includes, for example, setting a state in which no instruction is input even if the user operates the operation unit, and setting a state in which it is difficult to input an instruction in such a manner as, for example, requesting long press of a switch by the user.

### BRIEF DESCRIPTION OF THE DRAWINGS

The present invention will become more fully understood from the detailed description given hereinbelow and the accompanying drawings which are given by way of illustration only, and thus are not limitative of the present invention, and wherein:

FIG. 1 is a schematic diagram of a wrist-type sphygmomanometer according to an embodiment of the present invention.

FIG. 2 is a schematic view illustrating a state in which the wrist-type sphygmomanometer illustrated in FIG. 1 is wound around a left wrist.

FIG. 3 is a block diagram of the wrist-type sphygmomanometer illustrated in FIG. 1.

FIG. 4 is a flowchart of automatic blood pressure measurement performed by the wrist-type sphygmomanometer illustrated in FIG. 1.

FIG. 5 is a diagram illustrating a switching condition between a normal blood pressure measurement mode and an automatic blood pressure measurement mode in the wrist-type sphygmomanometer illustrated in FIG. 1.

FIG. 6 is a flowchart of automatic blood pressure measurement mode cancellation processing for canceling the automatic blood pressure measurement mode.

### MODE(S) FOR CARRYING OUT THE INVENTION

Hereinafter, an embodiment of a wrist-type sphygmomanometer according to the present invention will be described with reference to the accompanying drawings.

### [Wrist-Type Sphygmomanometer]

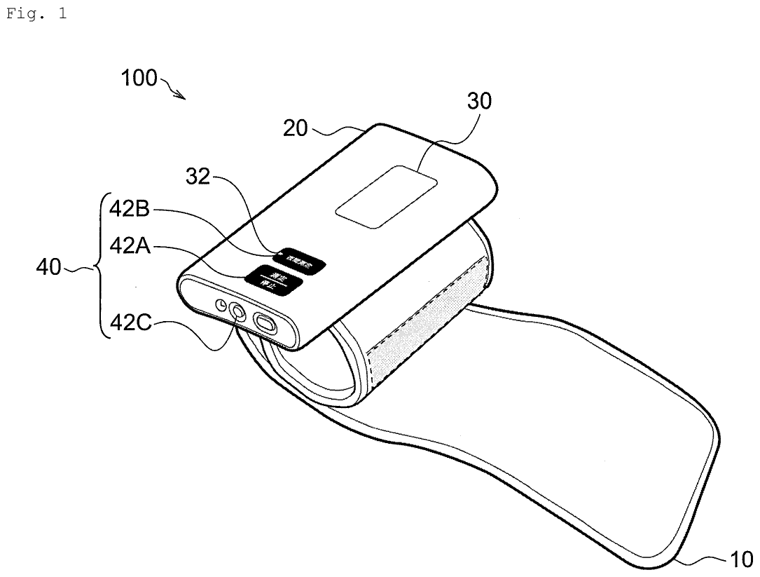

FIG. 1 illustrates a schematic configuration of a wrist-type sphygmomanometer (hereinafter, appropriately referred to as a “sphygmomanometer”) 100 according to an embodiment of the present invention. As will be described later, the sphygmomanometer 100 has a normal blood pressure measurement mode in which blood pressure measurement is started immediately after a blood pressure measurement switch is turned on, and an automatic blood pressure measurement mode in which blood pressure measurement is started according to a predetermined schedule.

### [Configuration of Wrist-Type Sphygmomanometer]

As illustrated in FIG. 1, the sphygmomanometer 100 includes a cuff 10 for blood pressure measurement wound around a measured part of a subject and a sphygmomanometer main body 20 integrally attached to the cuff 10.

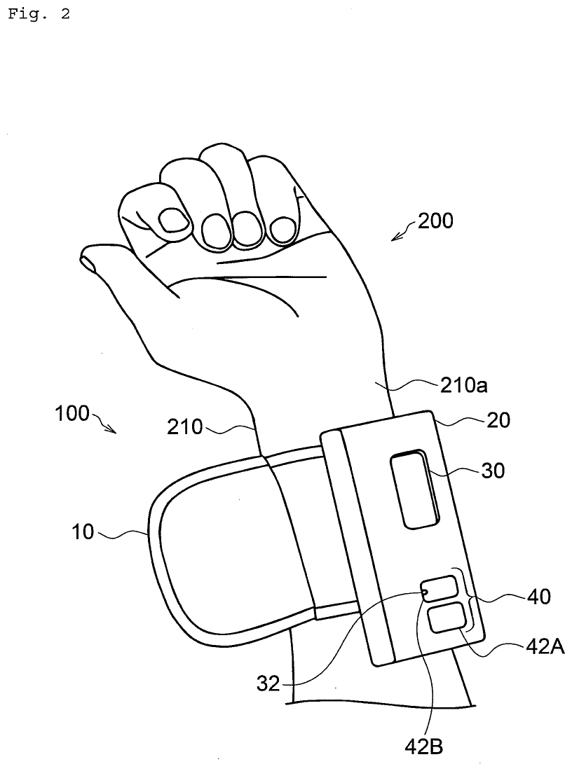

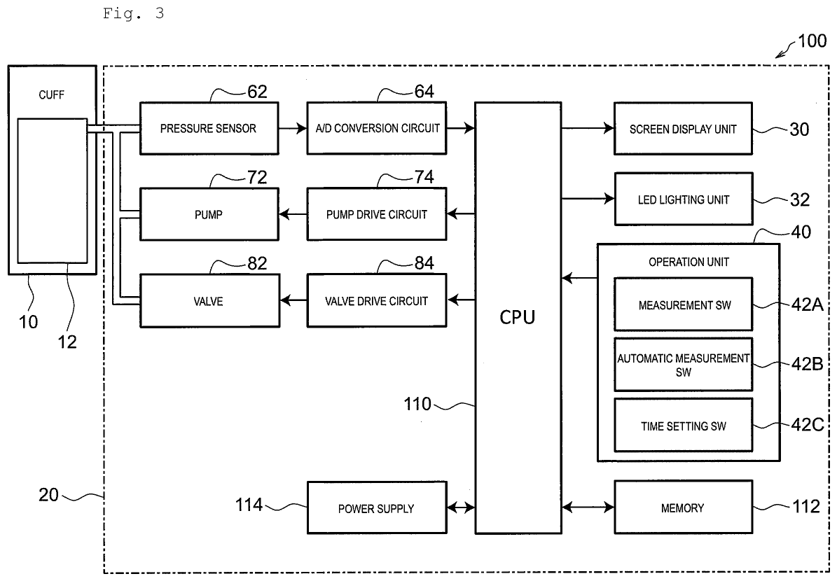

As illustrated in FIG. 2, the sphygmomanometer 100 according to the embodiment is a wrist-type sphygmomanometer. Therefore, the cuff 10 has an elongated band-like shape so as to be wound around a left wrist 210 of a subject 200, for example. The cuff 10 includes an air bag 12 (see FIG. 3) for compressing the left wrist 210. Note that, in order to always maintain the cuff 10 in an annular shape, a curler (not illustrated) having appropriate flexibility may be provided in the cuff 10.

The sphygmomanometer main body 20 is integrally attached to a substantially central portion in the longitudinal direction of the cuff 10 having a band-like shape. In the embodiment, a part to which the sphygmomanometer main body 20 is attached is supposed to correspond to a palmar surface (surface on the palm side) 210a of the left wrist 210.

The sphygmomanometer main body 20 has a flat substantially rectangular parallelepiped shape along an outer peripheral surface of the cuff 10, and is formed small and thin so as not to disturb the sleep of the subject 200. A corner portion connecting an upper surface of the sphygmomanometer main body 20 illustrated in FIG. 1 and a side surface surrounding the upper surface is chamfered in a curved shape.

As illustrated in FIG. 1, a screen display unit 30 forming a display screen is provided on an upper surface on the side farthest from the left wrist 210 in an outer surface of the sphygmomanometer main body 20. An operation unit 40 for inputting an instruction from the subject 200 is provided on the upper surface and a side surface on the near side in the diagram.

In the embodiment, the screen display unit 30 includes a liquid crystal display (LCD), and is configured to display predetermined information, for example, maximal blood pressure (unit; mmHg), minimal blood pressure (unit; mmHg), pulse (unit; beats per minute) according to a control signal from a central processing unit (CPU) 110 described later. Note that the screen display unit 30 may be either an organic EL display or a light emitting diode (LED).

The operation unit 40 includes a plurality of buttons or switches operated by the subject 200. In the embodiment, the operation unit 40 includes a blood pressure measurement switch (normal blood pressure measurement operation unit) 42A for the subject 200 to input a blood pressure measurement instruction for the normal blood pressure measurement mode, an automatic measurement switch (automatic blood pressure measurement mode operation unit) 42B for the subject 200 to input a blood pressure measurement instruction for the automatic blood pressure measurement mode, and a clock setting switch (clock setting instruction operation unit) 42C for the subject 200 to input a clock setting instruction for performing clock setting of the sphygmomanometer main body 20. In a case where the sphygmomanometer 100 is set in the normal blood pressure measurement mode or in a case where the sphygmomanometer 100 is set in the automatic blood pressure measurement mode, the blood pressure measurement switch 42A functions as a switch that stops blood pressure measurement being executed when the switch is pressed during the blood pressure measurement.

In description below, “normal blood pressure measurement” refers to blood pressure measurement started immediately after the blood pressure measurement switch 42A is turned on. Further, in description below, “automatic blood pressure measurement” means blood pressure measurement automatically performed according to a predetermined schedule after a blood pressure measurement instruction is input through the automatic measurement switch 42B, and is performed, for example, during sleep of the subject 200. The blood pressure measurement performed according to a predetermined schedule is, for example, blood pressure measurement performed at a predetermined time such as 1:00, 2:00, or 3:00 in the middle of the night, or blood pressure measurement performed at, for example, two hour intervals after the automatic measurement switch 42B is pressed.

In the embodiment, all of the blood pressure measurement switch 42A, the automatic measurement switch 42B, and the clock setting switch 42C are a momentary type (self-return type) switch, and is configured to be in an on state only while being pressed down, and to be returned to an off state when released from pressing.

As illustrated in FIGS. 1 and 2, an LED lighting unit (display device) 32 is provided at an end portion of the automatic measurement switch 42B. When the mode of the sphygmomanometer 100 is switched to the automatic blood pressure measurement mode, the LED lighting unit 32 keeps lighting for ten seconds (display time) when the automatic measurement switch 42B is pressed down. Further, the time during which the LED lighting unit 32 continues to light up is not limited to this content, and may be, for example, five seconds or 20 seconds.

FIG. 3 illustrates a block configuration of the sphygmomanometer 100.

The air bag 12 included in the cuff 10 described above and various fluid control devices (described below) included in the sphygmomanometer main body 20 are connected by an air pipe 50 in a manner that fluid can circulate.

In addition to the screen display unit 30, the LED lighting unit 32, and the operation unit 40 described above, the sphygmomanometer main body 20 includes the CPU 110 as a control unit, a memory 112 as a storage unit, a power supply unit 114, a pressure sensor 62, a pump 72, and a valve 82. The sphygmomanometer main body 20 includes an A/D conversion circuit 64 that converts output of the pressure sensor 62 from an analog signal to a digital signal, a pump drive circuit 74 that drives the pump 72, and a valve drive circuit 84 that drives the valve 82. The pressure sensor 62, the pump 72, and the valve 82 are connected to the air bag 12 in a manner that fluid can circulate through the air pipe 50.

The memory 112 stores a program for controlling the sphygmomanometer 100, data used for controlling the sphygmomanometer 100, setting data for setting various functions of the sphygmomanometer 100, data of a measurement result of a blood pressure value, and the like. The memory 112 is also used as a work memory that temporarily stores various types of information during program execution. In particular, the sphygmomanometer memory 112 according to the embodiment is configured as a program storage unit, and stores a normal blood pressure measurement program and an automatic blood pressure measurement program for calculating blood pressure by the oscillometric method described later.

The CPU 110 is configured to control operation of the entire sphygmomanometer 100. Specifically, the CPU 110 is configured as a pressure control unit that drives the pump 72 or the valve 82 according to a program for controlling the sphygmomanometer 100 stored in the memory 112, a restriction unit that restricts functions of the blood pressure measurement switch 42A, the automatic measurement switch 42B, and the clock setting switch 42C provided on the operation unit 40 when the mode of the sphygmomanometer 100 is switched to the automatic blood pressure measurement mode, and a measurement implementation unit that performs blood pressure measurement by a normal blood pressure measurement program or an automatic blood pressure measurement program to be described later. The CPU 110 also displays a blood pressure value obtained by performing blood pressure measurement on the screen display unit 30 and stores the blood pressure value in the memory 112. Further, when the mode of the sphygmomanometer 100 is switched to the automatic blood pressure measurement mode, and the automatic measurement switch 42B is pressed down, the CPU 110 keeps the LED lighting unit 32 being lit for ten seconds.

In the embodiment, when the clock setting switch 42C is pressed down, the CPU 110 performs clock setting of the sphygmomanometer main body 20. Specifically, the CPU 110 advances the clock of the sphygmomanometer 100 when the blood pressure measurement switch 42A continues to be pressed down after the clock setting switch 42C is pressed down, and returns the clock of the sphygmomanometer 100 when the automatic measurement switch 42B continues to be pressed down. After the above, when the clock setting switch 42C is pushed down again, the CPU 110 sets an adjusted clock as described above. In the automatic blood pressure measurement mode, the CPU 110 refers to this clock to determine whether or not it is a measurement time set in the schedule described above.

The power supply unit 114 includes a secondary battery, and is configured to supply power to each unit of the CPU 110, the pressure sensor 62, the pump 72, the valve 82, the screen display unit 30, the LED lighting unit 32, the memory 112, the A/D conversion circuit 64, the pump drive circuit 74, and the valve drive circuit 84. The power supply unit 114 is also configured to be able to switch between on and off states. For example, when power supply is in an off state, and the blood pressure measurement switch (power supply instruction operation unit) 42A is continuously pressed down for three seconds or more, the state is switched to an on state. Further, in the normal blood pressure measurement mode, when power supply is in the on state, and the blood pressure measurement switch (power supply instruction operation unit) 42A is continuously pressed down for three seconds or more, the state is switched to the off state.

The pump 72 is configured to supply air as fluid to the air bag 12 through the air pipe 50 in order to increase pressure (hereinafter, appropriately referred to as “cuff pressure”) in the air bag 12 built in the cuff 10. The valve 82 is configured to discharge air in the air bag 12 through the air pipe 50 by being opened or hold the cuff pressure by being closed in order to control the cuff pressure. The pump drive circuit 74 is configured to drive the pump 72 based on a control signal provided from the CPU 110. The valve drive circuit 84 is configured to open and close the valve 82 based on a control signal provided from the CPU 110.

The pressure sensor 62 and the A/D conversion circuit 64 are configured to detect the cuff pressure. The pressure sensor 62 in the embodiment is a piezoresistive pressure sensor, and detects and outputs the cuff pressure of the air bag 12 as electric resistance due to a piezoresistive effect. The A/D conversion circuit 64 converts output (electric resistance) of the pressure sensor 62 from an analog signal to a digital signal and outputs the converted signal to the CPU 110. In the embodiment, the CPU 110 acquires the cuff pressure according to the electric resistance output from the pressure sensor 62.

### [Blood Pressure Measurement Program]

The blood pressure measurement program calculates blood pressure of the subject 200 with the sphygmomanometer main body 20 attached to the left wrist 210. The blood pressure measurement program includes a normal blood pressure measurement program and an automatic blood pressure measurement program. The normal blood pressure measurement program assumes that the subject 200 sits on a chair or the like and keeps the left wrist 210 to which the sphygmomanometer main body 20 is attached at the same height as the heart of the subject 200. The automatic blood pressure measurement program assumes that the subject 200 lies on a bed or the like, and the left wrist 210 to which the sphygmomanometer main body 20 is attached is placed at a position lower than the heart of the subject 200. It is known that different blood pressure values are calculated when a relationship between the height of the sphygmomanometer main body 20 and the height of the heart of the subject 200 is different. For this reason, in the normal blood pressure measurement program and the automatic blood pressure measurement program, a parameter used for blood pressure calculation is adjusted in advance in consideration of a relationship between the height of the sphygmomanometer main body 20 and the height of the heart of the subject 200 assumed by the programs.

When performing the normal blood pressure measurement program or the automatic blood pressure measurement program, the CPU 110 obtains a pulse wave signal from a fluctuation component of a pulse wave included in the cuff pressure obtained by the pressure sensor 62, and calculates a blood pressure value (maximal blood pressure and minimal blood pressure) using the programs stored in the memory 112.

### [Automatic Blood Pressure Measurement Mode]

The automatic blood pressure measurement will be described. In a state in which the cuff 10 of the sphygmomanometer 100 is wound around the left wrist 210 of the subject 200 and the power supply is turned on (normal blood pressure measurement mode), when the subject 200 who is not sleeping presses the automatic measurement switch 42B of the sphygmomanometer main body 20 once, an instruction to make a transition to the automatic blood pressure measurement mode (automatic blood pressure measurement mode instruction) is output to the CPU 110.

After the above, the automatic blood pressure measurement program is performed according to a predetermined schedule.

In the embodiment, in a schedule of the automatic blood pressure measurement, the automatic blood pressure measurement program is performed at a time point at which predetermined time (for example, four hours) elapses after the automatic measurement switch 42B is pressed, and, as necessary, every certain period of time (for example, two hours) elapses during a period from a current time point to a predetermined time (for example, 7:00 AM). In a mode in which the time at which the automatic blood pressure measurement is performed is calculated based on a time point when the automatic measurement switch 42B is pressed, the automatic blood pressure measurement program includes a program (not illustrated) that determines a measurement time, and the measurement time is determined based on the time determination program.

The performing schedule of the automatic blood pressure measurement is not limited to the above, and the automatic blood pressure measurement program may be set to be performed at a predetermined reservation time, for example, 1:00, 2:00, and 3:00 in the morning.

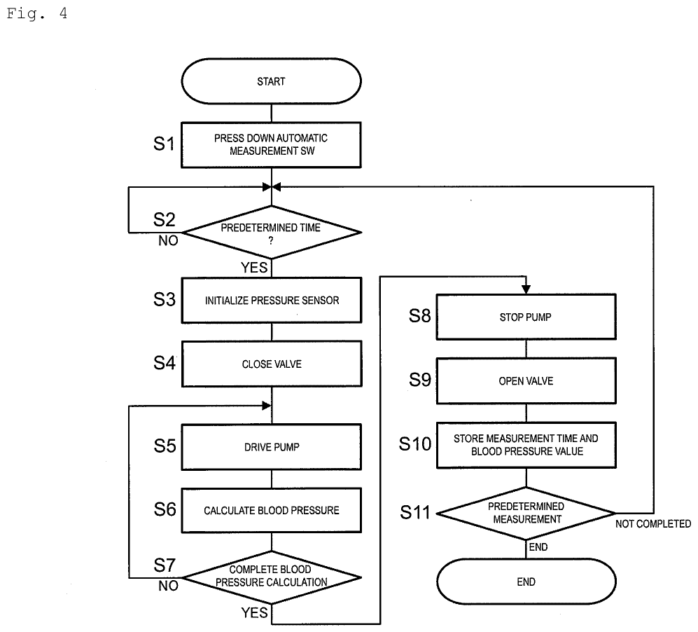

FIG. 4 illustrates an operation process when the subject 200 performs the automatic blood pressure measurement with the sphygmomanometer 100. During this automatic blood pressure measurement, the subject 200 wearing the sphygmomanometer 100 on the left wrist 210 keeps being in a state of lying on a bed or the like.

In this state, as shown in Step S1 of FIG. 4, when the subject 200 presses the automatic measurement switch 42B provided on the sphygmomanometer main body 20 to input a blood pressure measurement instruction for the automatic blood pressure measurement mode, the CPU 110 determines whether or not it is the measurement time set in the schedule of the automatic blood pressure measurement (Step S2), and if it is not the measurement time set in the schedule (when the processing proceeds to NO in Step S2), the CPU 110 waits until the measurement time.

When the measurement time comes (when the processing proceeds to YES in Step S2), the CPU 110 initializes the pressure sensor 62 (Step S3). Specifically, the CPU 110 initializes a processing memory area, stops the pump 72, and performs 0-mmHg adjustment (sets the atmospheric pressure to 0 mmHg) of the pressure sensor 62 in a state where the valve 82 is opened.

Next, the CPU 110 closes the valve 82 via the valve drive circuit 84 (Step S4), and then drives the pump 72 via the pump drive circuit 74 to start pressurization of the cuff 10 (air bag 12) (Step S5). At this time, the CPU 110 controls a pressurization rate of the cuff pressure, which is the pressure in the air bag 12, based on output of the pressure sensor 62 while supplying air from the pump 72 to the air bag 12 through the air pipe 50.

Next, in Step S6, the CPU 110 calculates blood pressure values (maximal blood pressure and minimal blood pressure) using the above-described automatic blood pressure measurement program stored in the memory 112 based on a pulse wave signal acquired at this time point.

At this time point, when the blood pressure value cannot be calculated yet due to lack of data (in a case where the processing proceeds to NO in Step S7), the CPU 110 repeats the processing of Steps S5 and S6 unless the cuff pressure reaches an upper limit pressure (set to, for example, 300 mmHg for safety).

When a blood pressure value is calculated (in a case where the processing proceeds to YES in Step S7), the CPU 110 stops the pump 72 (Step S8), opens the valve 82 (Step S9), and performs control to discharge air in the cuff 10 (air bag 12).

After the above, the CPU 110 displays the calculated blood pressure value on the screen display unit 30, and performs control to store the sphygmomanometer in the memory 112 (Step S10).

When one time of the blood pressure measurement set in the above-described schedule is completed, the CPU 110 determines whether or not all the blood pressure measurements set in the above-described schedule are completed (Step S11). In a case where the blood pressure measurement set in the above-described schedule is still scheduled (when the processing proceeds to “not completed” in Step S11), the CPU 110 returns to Step S2 and determines whether or not it is a next measurement time set in the above-described schedule. If it is not the measurement time (when the processing proceeds to NO in Step S2), the CPU 110 waits until the measurement time comes.

When the next measurement time set in the schedule is reached (when the processing proceeds to YES in Step S2), the CPU 110 repeats the processing of Steps S3 to S10, and again determines whether or not all the blood pressure measurements set in the schedule are completed in Step S11.

When all the blood pressure measurements set in the schedule described above are completed (when the processing proceeds to “end” in Step S11), the CPU 110 ends the automatic blood pressure measurement.

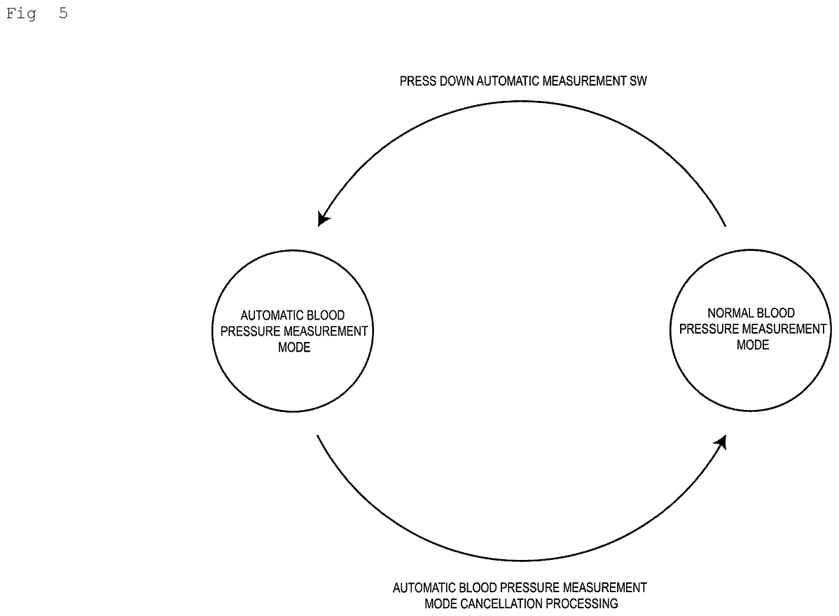

FIG. 5 illustrates a condition for the CPU 110 to switch the mode of the sphygmomanometer 100 between the normal blood pressure measurement mode and the automatic blood pressure measurement mode.

As described above, when the sphygmomanometer 100 is in the normal blood pressure measurement mode and the automatic measurement switch 42B is pressed, the mode of the sphygmomanometer 100 is switched to the automatic blood pressure measurement mode, that is, a state where the automatic blood pressure measurement mode is set is obtained. After the above, the CPU 110 performs the automatic blood pressure measurement according to the operation process illustrated in FIG. 4.

In the embodiment, when the sphygmomanometer 100 is set in the automatic blood pressure measurement mode, even if the blood pressure measurement switch 42A is pressed, the CPU (restriction unit) 110 restricts the blood pressure measurement according to the blood pressure measurement instruction by the blood pressure measurement switch 42A performed by the sphygmomanometer 100. In this manner, when the sphygmomanometer 100 is set in the automatic blood pressure measurement mode, it is possible to avoid a situation in which unscheduled blood pressure measurement is performed by interruption.

Further, the CPU 110 restricts the power supply being in the off state when the sphygmomanometer 100 is set in the automatic blood pressure measurement mode. That is, even if the blood pressure measurement switch 42A is continuously pressed for three seconds or more, the CPU 110 restricts switching of the power supply unit 114 of the sphygmomanometer 100 to the off state. In this manner, when the sphygmomanometer 100 is set in the automatic blood pressure measurement mode, it is possible to avoid a situation in which the power supply unit 114 is erroneously turned off. Therefore, the automatic blood pressure measurement is reliably performed. Note that the restriction of the function of switching the power supply 114 to the off state does not impair the function of stopping the blood pressure measurement being executed when the blood pressure measurement switch 42A is pressed during the blood pressure measurement.

Furthermore, when the sphygmomanometer 100 is in a state of being set in the automatic blood pressure measurement mode, the CPU 110 restricts performance of the clock setting by pressing of the clock setting switch 42C.

In this manner, when the sphygmomanometer 100 is set in the automatic blood pressure measurement mode, it is possible to avoid a situation in which the clock setting is erroneously performed and the blood pressure measurement is started at a time different from that on the schedule.

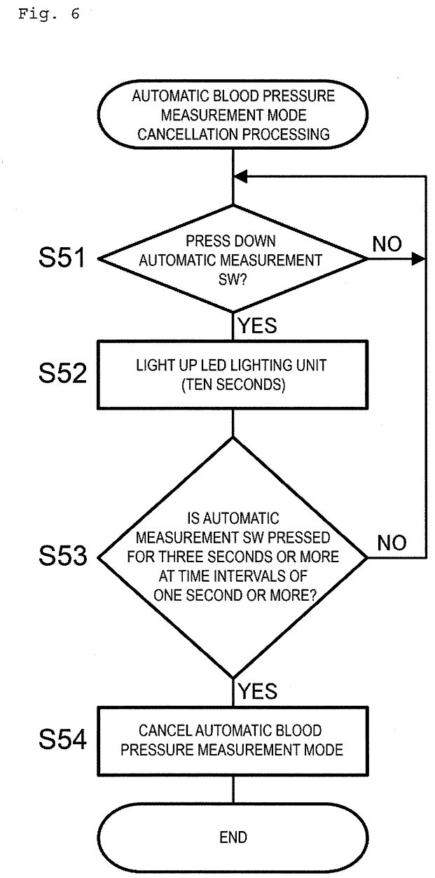

The automatic blood pressure measurement mode is canceled (in this example, transition is made to the normal blood pressure measurement mode) by the automatic blood pressure measurement mode cancellation processing illustrated in FIG. 6. That is, when the automatic measurement switch 42B is pressed as shown in Step S51 (when the processing proceeds to YES in Step S51) while the sphygmomanometer 100 is set in the automatic blood pressure measurement mode, the LED lighting unit 32 keeps lighting for ten seconds (display time) as shown in Step S52. The lighting of the LED lighting unit 32 provided at the end portion of the automatic measurement switch 42B means that the mode of the sphygmomanometer 100 is the automatic blood pressure measurement mode. While the LED lighting unit 32 continues to light up, when the automatic measurement switch 42B is continuously pressed for three seconds (input time) or more at time intervals of one second or more (when the processing proceeds to YES in Step S53) as shown in Step S53, a cancellation instruction for the automatic blood pressure measurement mode for switching the mode of the sphygmomanometer 100 from the automatic blood pressure measurement mode is output to the CPU 110, and the automatic blood pressure measurement mode is canceled as shown in Step S54 (in this example, transition is made to the normal blood pressure measurement mode). Therefore, when the sphygmomanometer 100 is set in the automatic blood pressure measurement mode, the automatic blood pressure measurement mode is canceled with the above-described operation (Steps S51 and S53) intentionally performed by the subject 200 as a requirement. In this manner, it is possible to avoid a situation in which the automatic blood pressure measurement mode is erroneously canceled.

Furthermore, when the sphygmomanometer 100 is set in the automatic blood pressure measurement mode, the CPU (restriction unit) 110 restricts cancellation of the automatic blood pressure measurement mode in processing other than the above-described automatic blood pressure measurement mode cancellation processing (FIG. 6). In this manner, when the sphygmomanometer 100 is set in the automatic blood pressure measurement mode, it is possible to avoid a situation in which the automatic blood pressure measurement mode is erroneously canceled. Therefore, the automatic blood pressure measurement is reliably performed.

As described above, according to the sphygmomanometer 100, when the sphygmomanometer 100 is set in the automatic blood pressure measurement mode, it is possible to avoid a situation in which the automatic blood pressure measurement is not properly performed due to unintended operation. Therefore, the automatic blood pressure measurement is properly performed.

Since the sphygmomanometer 100 is of a type that presses a wrist (the left wrist 210 in the embodiment, which may be the right wrist) as a measured part, it is expected that the sphygmomanometer 100 is less likely to disturb the sleep of the subject 200 than that of a type that presses an upper arm (Imai et al., “Development and evaluation of a home nocturnal blood pressure monitoring system using a wrist-cuff device”, Blood Pressure Monitoring 2018, 23, 318-326). Therefore, the sphygmomanometer 100 is suitable for automatic (during-sleep) blood pressure measurement.

Further, since the sphygmomanometer 100 is integrally and compactly configured as a wrist-type sphygmomanometer, the subject 200 can easily handle the sphygmomanometer 100.

### Other Embodiments

In the above-described embodiment, the CPU 110 calculates blood pressure in a pressurization process of the cuff 10 (air bag 12), but may calculate blood pressure in a depressurization process of the cuff.

In the above-described embodiment, the sphygmomanometer 100 includes the blood pressure measurement switch 42A to which a normal blood pressure measurement instruction is input and the automatic measurement switch 42B to which an automatic blood pressure measurement instruction is input. However, for example, a signal receiving unit of the sphygmomanometer may receive an instruction from a smartphone or the like existing outside the sphygmomanometer via wireless communication, and a signal received by the signal receiving unit may be replaced with a signal output from the normal blood pressure measurement switch or the automatic measurement switch to the CPU.

In the above-described embodiment, the sphygmomanometer 100 is configured such that the blood pressure measurement switch 42A outputs a signal of a normal blood pressure measurement instruction to the CPU 110, and the automatic measurement switch 42B outputs a signal of an automatic blood pressure measurement instruction to the CPU 110. However, for example, the sphygmomanometer may be configured such that the blood pressure measurement switch is pressed once to output a signal of a normal blood pressure measurement instruction to the CPU, and the blood pressure measurement switch is pressed twice within a certain period of time to output a signal of an automatic blood pressure measurement instruction to the CPU.

In the above-described embodiment, the sphygmomanometer main body 20 is integrally attached to the cuff 10, but may be provided separately from the cuff and connected to the cuff 10 (air bag 12) via a flexible air tube in such a manner that fluid can circulate.

In the above-described embodiment, the normal blood pressure measurement program, the automatic blood pressure measurement program, and the process of these are stored in the memory 112 as software, but may be recorded on a non-transitory medium such as a compact disc (CD), a digital versatile disc (DVD), or a flash memory. By installing software recorded in the above-described medium in a substantial computer device such as a personal computer, a personal digital assistant (PDA), or a smartphone, the above-described program and process can be executed by the computer device.

As described above, a sphygmomanometer of the present disclosure has an automatic blood pressure measurement mode for automatically starting blood pressure measurement according to a predetermined schedule, the sphygmomanometer comprising:

a sphygmomanometer main body configured to be mounted on a measured part; and

a plurality of operation units to which instructions different from each other are input, the plurality of operation units being provided on the sphygmomanometer main body, wherein

the operation unit includes an automatic blood pressure measurement mode operation unit to which an automatic blood pressure measurement mode instruction is input for switching a mode to the automatic blood pressure measurement mode, and

the sphygmomanometer includes a restriction unit that restricts a function of the operation unit when the sphygmomanometer is set in the automatic blood pressure measurement mode as a result of the automatic blood pressure measurement mode operation unit being operated.

In the present description, the “operation unit” typically refers to a switch provided on the main body of the sphygmomanometer, and receives switching on by the user (mainly a subject) as an instruction. Note that operation itself of the sphygmomanometer may be performed by an instruction from a smartphone or the like existing outside the sphygmomanometer via wireless communication.

To “restrict” a function of the operation unit includes, for example, setting a state in which no instruction is input even if the user operates the operation unit, and setting a state in which it is difficult to input an instruction in such a manner as, for example, requesting long press of a switch by the user.

In the sphygmomanometer of the present disclosure, for example, in a state where the sphygmomanometer main body is mounted on the measured part, a subject inputs an automatic blood pressure measurement mode instruction for switching a mode to the automatic blood pressure measurement mode for automatically starting blood pressure measurement according to a predetermined schedule by the automatic blood pressure measurement mode operation unit included in a plurality of the operation units to which instructions different from each other are input. When the sphygmomanometer is set in the automatic blood pressure measurement mode, the restriction unit restricts a function of the operation unit. In this manner, when the sphygmomanometer is set in the automatic blood pressure measurement mode, it is possible to avoid a situation in which the automatic blood pressure measurement is not properly performed due to unintended operation. Therefore, the automatic blood pressure measurement is properly performed.

The present disclosure provides the sphygmomanometer according to one embodiment, wherein

the operation unit includes a normal blood pressure measurement operation unit to which a blood pressure measurement instruction is input for a normal blood pressure measurement mode in which the blood pressure measurement is performed according to an input of the blood pressure measurement instruction, and

the restriction unit restricts performance of the blood pressure measurement according to the blood pressure measurement instruction by the normal blood pressure measurement operation unit when the sphygmomanometer is set in the automatic blood pressure measurement mode.

In the sphygmomanometer of this embodiment, when the sphygmomanometer is set in the automatic blood pressure measurement mode, the restriction unit restricts performance of the blood pressure measurement according to the blood pressure measurement instruction by the normal blood pressure measurement operation unit (input of a blood pressure measurement instruction for the normal blood pressure measurement mode). In this manner, when the sphygmomanometer is set in the automatic blood pressure measurement mode, it is possible to avoid a situation in which unscheduled blood pressure measurement is performed by interruption.

The present disclosure provides the sphygmomanometer according to one embodiment, wherein

the operation unit includes a power supply instruction operation unit to which an instruction to turn on or off power supply of the sphygmomanometer is input, and

the restriction unit restricts turning off of the power supply of the sphygmomanometer by operation of the power supply instruction operation unit when the sphygmomanometer is set in the automatic blood pressure measurement mode.

In the sphygmomanometer of this embodiment, when the sphygmomanometer is set in the automatic blood pressure measurement mode, the restriction unit restricts turning off of the power supply of the sphygmomanometer by operation of the power supply instruction operation unit (input of an instruction to turn on or off the power supply of the sphygmomanometer). In this manner, when the sphygmomanometer is set in the automatic blood pressure measurement mode, it is possible to avoid a situation in which the power supply is erroneously turned off. Therefore, the automatic blood pressure measurement is reliably performed.

The present disclosure provides the sphygmomanometer according to one embodiment, wherein

the restriction unit restricts cancellation of the automatic blood pressure measurement mode when the sphygmomanometer is set in the automatic blood pressure measurement mode.

In the sphygmomanometer of this embodiment, when the sphygmomanometer is set in the automatic blood pressure measurement mode, the restriction unit restricts cancellation of the automatic blood pressure measurement mode. In this manner, when the sphygmomanometer is set in the automatic blood pressure measurement mode, it is possible to avoid a situation in which the automatic blood pressure measurement mode is erroneously canceled. Therefore, the automatic blood pressure measurement is reliably performed.

The present disclosure provides the sphygmomanometer according to one embodiment, wherein

the sphygmomanometer main body includes a display device, and

the restriction unit

causes the display device to maintain a display indicating that the sphygmomanometer is set in the automatic blood pressure measurement mode until a predetermined display time elapses by the automatic blood pressure measurement mode operation unit being operated when the sphygmomanometer is set in the automatic blood pressure measurement mode, and

cancels the automatic blood pressure measurement mode by the automatic blood pressure measurement mode operation unit being continuously operated for longer than a predetermined input time at a predetermined time interval when the display device maintains the display.

Here, the “display device” may be, for example, a light emitting diode (LED) provided on the main body of the sphygmomanometer, and may be configured to indicate that the sphygmomanometer is set in the automatic blood pressure measurement mode by lighting up, or may be a liquid crystal display (LCD), and may be configured to indicate that the sphygmomanometer is set in the automatic blood pressure measurement mode by displaying text “automatic blood pressure measurement mode ON”.

The “predetermined display time” is, for example, ten seconds.

The “predetermined time interval” is, for example, one second.

Furthermore, the “predetermined input time” is, for example, time during which switching on is continuously performed by the user in a case where the automatic blood pressure measurement mode operation unit is a switch, and is, for example, three seconds.

In the sphygmomanometer of this embodiment, when the sphygmomanometer is set in the automatic blood pressure measurement mode, for example, as the subject operates the automatic blood pressure measurement mode operation unit, the restriction unit causes the display device to maintain the display (display indicating that the sphygmomanometer is set in the automatic blood pressure measurement mode) until predetermined display time elapses. At this time, when the subject continues to operate the automatic blood pressure measurement mode operation unit longer than the predetermined input time at the predetermined time intervals, the restriction unit cancels the automatic blood pressure measurement mode. Therefore, when the sphygmomanometer is set in the automatic blood pressure measurement mode, the automatic blood pressure measurement mode is canceled only by operation intentionally performed by the subject. In this manner, it is possible to avoid a situation in which the automatic blood pressure measurement mode is erroneously canceled.

The present disclosure provides the sphygmomanometer according to one embodiment, wherein

the operation unit includes a clock setting instruction operation unit to which a clock setting instruction is input for performing a clock setting of the sphygmomanometer, and

the restriction unit restricts performance of the clock setting by operation of the clock setting instruction operation unit when the sphygmomanometer is set in the automatic blood pressure measurement mode.

Here, the “clock setting” refers to advancing or returning the clock of the sphygmomanometer serving as a reference of a schedule of the blood pressure measurement performed in the automatic blood pressure measurement mode.

In the sphygmomanometer of this embodiment, when the sphygmomanometer is set in the automatic blood pressure measurement mode, the restriction unit restricts performance of the clock setting by operation of the clock setting instruction operation unit (input of a clock setting instruction for performing clock setting of the sphygmomanometer). In this manner, when the sphygmomanometer is set in the automatic blood pressure measurement mode, it is possible to avoid a situation in which the clock setting is erroneously performed and the blood pressure measurement is started at a time different from that on the schedule.

The present disclosure provides the sphygmomanometer according to one embodiment, wherein

the measured part is a wrist.

Since the sphygmomanometer of this embodiment is of a type that presses a wrist as the measured part, it is expected that the sphygmomanometer of this embodiment has less degree of disturbing the sleep of the subject than a type that presses an upper arm (Imai et al., “Development and evaluation of a home nocturnal blood pressure monitoring system using a wrist-cuff device”, Blood Pressure Monitoring 2018, 23, P 318-326). Therefore, this sphygmomanometer is suitable for automatic (during-sleep) blood pressure measurement.

The present disclosure provides the sphygmomanometer according to one embodiment, further comprising:

a blood pressure measuring cuff provided integrally with the sphygmomanometer main body, wherein

the sphygmomanometer main body includes a blood pressure measurement unit that temporarily presses the wrist by the blood pressure measuring cuff and performs the blood pressure measurement by an oscillometric method for detecting pressure in the cuff, the operation unit, and the restriction unit.

Here, the “blood pressure measuring unit” includes, for example, a pump that supplies fluid for pressurization to the blood pressure measuring cuff, a valve that exhausts fluid from the blood pressure measuring cuff, and an element that drives and controls the pump, valve, and the like.

The sphygmomanometer of this embodiment can be configured integrally and compactly. Therefore, handling by the user is convenient.

As clear from the above, according to the sphygmomanometer of the present invention, when the automatic blood pressure measurement mode is set, it is possible to avoid a situation in which the automatic blood pressure measurement is not properly performed due to unintended operation.

It is to be noted that the various embodiments described above can be appreciated individually within each embodiment, but the embodiments can be combined together. It is also to be noted that the various features in different embodiments can be appreciated individually by its own, but the features in different embodiments can be combined.

1. A sphygmomanometer having an automatic blood pressure measurement mode for automatically starting blood pressure measurement according to a predetermined schedule, the sphygmomanometer comprising:
a sphygmomanometer main body configured to be mounted on a measured part; and
a plurality of operation units to which instructions different from each other are input, the plurality of operation units being provided on the sphygmomanometer main body, wherein
the operation unit includes an automatic blood pressure measurement mode operation unit to which an automatic blood pressure measurement mode instruction is input for switching a mode to the automatic blood pressure measurement mode, and
the sphygmomanometer includes a restriction unit that restricts a function of the operation unit when the sphygmomanometer is set in the automatic blood pressure measurement mode as a result of the automatic blood pressure measurement mode operation unit being operated.

2. The sphygmomanometer according to claim 1, wherein
the operation unit includes a normal blood pressure measurement operation unit to which a blood pressure measurement instruction is input for a normal blood pressure measurement mode in which the blood pressure measurement is performed according to an input of the blood pressure measurement instruction, and
the restriction unit restricts performance of the blood pressure measurement according to the blood pressure measurement instruction by the normal blood pressure measurement operation unit when the sphygmomanometer is set in the automatic blood pressure measurement mode.

3. The sphygmomanometer according to claim 1, wherein
the operation unit includes a power supply instruction operation unit to which an instruction to turn on or off power supply of the sphygmomanometer is input, and
the restriction unit restricts turning off of the power supply of the sphygmomanometer by operation of the power supply instruction operation unit when the sphygmomanometer is set in the automatic blood pressure measurement mode.

4. The sphygmomanometer according to claim 1, wherein
the restriction unit restricts cancellation of the automatic blood pressure measurement mode when the sphygmomanometer is set in the automatic blood pressure measurement mode.

5. The sphygmomanometer according to claim 4, wherein
the sphygmomanometer main body includes a display device, and
the restriction unit
causes the display device to maintain a display indicating that the sphygmomanometer is set in the automatic blood pressure measurement mode until a predetermined display time elapses by the automatic blood pressure measurement mode operation unit being operated when the sphygmomanometer is set in the automatic blood pressure measurement mode, and
cancels the automatic blood pressure measurement mode by the automatic blood pressure measurement mode operation unit being continuously operated for longer than a predetermined input time at a predetermined time interval when the display device maintains the display.

6. The sphygmomanometer according to claim 1, wherein
the operation unit includes a clock setting instruction operation unit to which a clock setting instruction is input for performing a clock setting of the sphygmomanometer, and
the restriction unit restricts performance of the clock setting by operation of the clock setting instruction operation unit when the sphygmomanometer is set in the automatic blood pressure measurement mode.

7. The sphygmomanometer according to claim 1, wherein
the measured part is a wrist.

8. The sphygmomanometer according to claim 7, further comprising:
a blood pressure measuring cuff provided integrally with the sphygmomanometer main body, wherein
the sphygmomanometer main body includes a blood pressure measurement unit that temporarily presses the wrist by the blood pressure measuring cuff and performs the blood pressure measurement by an oscillometric method for detecting pressure in the cuff, the operation unit, and the restriction unit.
